(19) United States
(12) Patent Application Publication
(10) Pub. No.: US 2014/0213904 A1
(43) Pub. Date: Jul. 31, 2014

(54) INTRA-FRACTION MOTION MANAGEMENT SYSTEM

(71) Applicants: Rui Chen, Stockholm (SE); Malcolm Williams, Stockholm (SE)

(72) Inventors: Rui Chen, Stockholm (SE); Malcolm Williams, Stockholm (SE)

(73) Assignee: ELEKTA AB (PUBL), Stockholm (SE)

(21) Appl. No.: 14/240,185

(22) PCT Filed: Aug. 26, 2011
(86) PCT No.: PCT/EP2011/064743

§ 371 Date: Feb. 21, 2014

(51) Int. Cl. A61N 5/10; A61B 8/00; A61G 13/12; A61B 8/08

(52) U.S. Cl. CPC A61N 5/1049; A61B 8/0875; A61B 8/4483; A61N 5/1067; A61G 13/121; A61N 2005/1058; A61N 2005/1097
USPC 600/439; 600/1

(57) ABSTRACT

The present invention relates to the field of radiation therapy. In particular, the invention concerns systems and methods for monitoring intra-fraction motions of patients in connection with treatment cancer in radiation therapy system. Ultrasound waves are generated in a direction of a bone or a part of a bone of the patient being in a fixed relation to a cancer tumor to be treated by radiation therapy such that the generated ultrasound waves are reflected by the bone or part of bone using an ultrasonic transducer and sensing the reflected ultrasound waves at least one ultrasonic transducer. Time intervals between generated ultrasound waves and corresponding sensed reflected ultrasound signals are determined for the at least one ultrasonic transducer. Based on the time intervals, motions of the bone or part of bone are monitored using changes of the time intervals, wherein the motions indicate that the patient or a part of the patient has moved.

### FIELD OF THE INVENTION

The present invention relates to the field of radiation therapy. In particular, the invention concerns systems and methods for monitoring intra-fraction motions of patients in connection with treatment cancer in radiation therapy system.

### BACKGROUND OF THE INVENTION

The development of surgical techniques has made great progress over the years. For instance, for patients suffering from cancer tumors requiring surgery, non-invasive surgery is now available which is afflicted with very little trauma to the patient.

One system for non-invasive surgery is sold under the name of Leksell Gamma Knife®, which provides such surgery by means of gamma radiation. The radiation is emitted from a large number of fixed radioactive sources and is focused by means of collimators, i.e. passages or channels for obtaining a beam of limited cross section, towards a defined target or treatment volume. Each of the sources provides a dose of gamma radiation which is insufficient to damage intervening tissue. However, tissue destruction occurs where the radiation beams from all radiation sources intersect or converge, causing the radiation to reach tissue-destructive levels. The point of convergence is hereinafter referred to as the “focus point”. Such a radiation device is, for example, referred to and described in U.S. Pat. No. 4,780,898.

Another system for non-invasive surgery is a linear accelerator (LINAC) which also be used in stereotactic radiosurgery similar to that achieved using the gamma knife on targets within e.g. the brain. The linear accelerator is used to treat all parts/organs of the body. It delivers a uniform dose of high-energy x-ray to the region of the patient's tumor. These x-rays can destroy the cancer cells while sparing the surrounding normal tissue. The LINAC is used to treat all body sites with cancer and used in not only external beam radiation therapy, but also for Stereotactic Radiosurgery and Stereotactic Body Radiotherapy. The linear accelerator uses microwave technology (similar to that used for radar) to accelerate electrons in a part of the accelerator called the “wave guide”, then allows these electrons to collide with a heavy metal target. As a result of the collisions, high-energy x-rays are produced from the target. These high energy x-rays will be directed to the patient's tumor and shaped as they exit the machine to conform to the shape of the patient's tumor. The beam may be shaped either by blocks that are placed in the head of the machine or by a multileaf collimator that is incorporated into the head of the machine. The beam comes out of a part of the accelerator called a gantry, which rotates around the patient. The patient lies on a moveable treatment couch and lasers are used to make sure the patient is in the proper position. The treatment couch can move in many directions including up, down, right, left, in and out. Radiation can be delivered to the tumor from any angle by rotating the gantry and moving the treatment couch.

Stereotactic radiation surgery is a minimally invasive treatment modality that allows delivery of a large single dose of radiation to a specific intracranial target while sparing surrounding tissue. Unlike conventional fractionated radiation therapy, stereotactic radiation surgery does not rely on, or exploit, the higher radiation sensitivity of neoplastic lesions relative to normal brain (therapeutic ratio). Its selective destruction depends primarily on sharply focused high-dose radiation and a steep dose gradient away from the defined target. The biological effect is irreparable cellular damage and delayed vascular occlusion within the high-dose target volume. Because a therapeutic ratio is not required, traditionally radiation resistant lesions can be treated. Because destructive doses are used, however, any normal structure included in the target volume is subject to damage.

In radiation therapy system such as in a LINAC or Leksell Gamma Knife®, the head of a patient is immobilized in a stereotactic instrument which defines the location of the treatment volume in the head. Further, the patient is secured in a patient positioning unit which moves the entire patient so as to position the treatment volume in coincidence with the focus point of the radiation unit of the radiation therapy system. Consequently, in radiation therapy systems, such as a LINAC system or a Leksell Gamma Knife® system, it is of a high importance that the positioning unit which moves the patient so as to position the treatment volume in coincidence with the focus point of the radiation unit of the system is accurate and reliable. That is, the positioning unit must be capable of position the treatment volume in coincidence with the focus point at a very high precision. This high precision must also be maintained over time.

Hence, in order to obtain as favourable clinical effect as possible during the therapy and to avoid damages to the surrounding tissue is it of an utmost importance that the radiation reaches and hits the target, i.e. the treatment volume, with a high precision and thereby spares the healthy tissue being adjacent to and/or surrounding the treatment volume. To achieve this, the patient must be immobilized during a therapy session and, moreover, the position of the head, or the part of the patient being under treatment, must be the same in a therapy session as in a reference position, i.e. the position during the session when the pictures to create the therapy plan were captured by means of, for example, Computerized Tomography Imaging (CT-imaging). For example, when the treatment area or volume is a portion of tissue within the head of a patient, a stereotactic fixation unit generally constituting a head fixation frame which, for example, may be fixed to the skull of the patient, e.g. by fixation screws or the like, is used to immobilize the head of the patient. Another example used in connection with treatment of tumors in the head or spinal cord of the patient is a face and shoulder mask adapted to be placed over the face and shoulders of the patient to thereby keep the patient in a substantially fixed position relative to the positioning system.

It is of particular importance to secure that the patient does not move during the delivery of the radiation therapy when conducting radiation surgery of tumors in proximity to sensitive tissue, such as in treatment of spinal tumors. Thus, when treating tumors of the spine, one must consider the different and sensitive tissue types around the spinal column including neural tissue, meningeal tissue, bone, and cartilage. For example, damage to the neural tissue caused by the radiation may lead to irreparable damages such as partial paralysis.

In the different fixation devices used today it is not possible to immobilize that patient to such an extent that motions of body parts of the patient is completely eliminated or prevented. For example, even though the patient is fixated using a face and shoulder mask there is a posibility that a cervical vertebra is moved as a result of a small motion of the head. If a spinal tumor in the cervical region is treated, very small movements of a cervical vertebra may result in that the radiation is mainly or partly delivered to the surrounding tissue instead of to the tumor, which may result in severe damages to the surrounding tissue.

In light of this, there is a need within the art of radiation therapy for intra-fraction motion management (IFMM) systems and methods that enable detection and monitoring of very small motions of the patient and the treated body part with a high degree of accuracy and reliability during the therapy, for example, in connection with treatment of cervical spine cancer.

In the prior art, there exists a number of solutions for intra-fraction motion management (IFMM) based on, for example, X-ray imaging, optical imaging or invasive solutions. However, these prior art methods are associated with drawbacks. For example, imaging methods such as X-ray imaging or optical imaging require extensive image processing which may lead to complex and expensive solutions. X-ray imaging also exposes the patient for radiation, which may be injurious. Invasive solutions may be uncomfortable for the patient and may also be injurious for the patient. Furthermore, the prior art systems may have problems in withstanding the gamma radiation generated in, for example, a Perfexion® system (a radiation therapy system provided by the applicant). Further, the prior art systems are often bulky which makes it difficult to use them together with, for example, the Perfexion® system.

Thus, there is a need within the art of radiation therapy for improved systems and methods that enable intra-fraction motion detection with a high degree of accuracy and reliability so as to avoid or at least significantly reduce the risk of undesired damages to surrounding sensitive tissue, for example, in connection with treatment of cervical spine cancer.

### SUMMARY OF THE INVENTION

An object of the present invention is to provide improved systems and methods for intra-fraction motion detection with a high degree of accuracy and reliability so as to avoid or at least significantly reduce the risk of undesired damages to surrounding sensitive tissue, for example, in connection with treatment of cervical spine cancer.

Another object of the present invention is to provide improved systems and methods for intra-fraction motion detection that can withstand gamma radiation.

A further object of the present invention is to provide improved systems and methods for intra-fraction motion detection that easily can be integrated into or be used together with radiation therapy system such as the Perfexion® system.

Yet another object of the present invention is to provide improved systems and methods for intra-fraction motion detection that can be manufactured at a low cost.

Still another object of the present invention is to provide improved systems and methods for intra-fraction motion management that are user-friendly and hence are easy to use for the medical personnel handling the radiation therapy system.

Another object of the present invention is to provide improved systems and methods for intra-fraction motion management that are comfortable for the patient during use thereof.

A further object of the present invention is to provide improved systems and methods for intra-fraction motion management that are compatible with imaging methods such as Computerized Tomography Imaging (CT-imaging) or Cone Beam Computerized Tomography Imaging (CBCT-imaging).

Another object of the present invention is to provide improved systems and methods for intra-fraction motion management that utilizes very low energy levels for the detection.

These and other objects are achieved by providing systems and methods having the features defined in the independent claim. Preferred embodiments are defined in the dependent claims.

According to an aspect of the present invention, there is provided a method for monitoring intra-fraction motions of a patient in connection with treatment of cancer tumors of the patient in a radiation therapy system such as the Perfexion® system, which radiation therapy system comprises a radiation therapy unit having a fixed radiation focus point and a patient positioning system for positioning a treatment volume in a patient in relation to the fixed focus point in the radiation therapy unit along motional axes, for example, along three substantially orthogonal motional axes or along motional axes in a polar coordinate system.

The method comprises generating ultrasound waves in a direction of a bone or a part of a bone of the patient being in a fixed relation to the cancer tumor such that the generated ultrasound waves are reflected by the bone or part of bone using an ultrasonic transducer and sensing the reflected ultrasound waves at least one ultrasonic transducer.

Time intervals between generated ultrasound waves and corresponding sensed reflected ultrasound signals are determined for the at least one ultrasonic transducer. Based on the time intervals, motions of the bone or part of bone are monitored using changes of the time intervals, wherein the motions indicate that the patient or a part of the patient has moved.

According to another aspect of the present invention, there is provided a system for monitoring intra-fraction motions of a patient in connection with treatment of cancer tumors of the patient in a radiation therapy system such as the Perfexion® system. The radiation therapy system comprises a radiation therapy unit having a fixed radiation focus point and a positioning system for positioning a treatment volume in a patient in relation to the fixed focus point in the radiation therapy unit along motional axes, for example, along three substantially orthogonal motional axes or along motional axes in a polar coordinate system.

The intra-fraction motion detection system comprises at least one ultrasonic transducer positioned relative to the positioning system to generate ultrasound waves in a direction of a bone or a part of a bone being in a fixed relation to the cancer tumor such that the generated ultrasound waves are reflected by the bone or part of bone, wherein a sensor of the at least one ultrasonic transducer is configured to sense the reflected ultrasound waves.

A time interval determining module is configured to determine the time intervals between generated ultrasound waves and corresponding sensed reflected ultrasound signals for the at least one ultrasonic transducer.

A monitoring module is configured to detect changes in the time intervals and to monitor motions of the bone or part of bone using the detected changes of the time intervals, wherein a motion indicates that the patient or a part of the patient has moved.

Thus, the present invention is based on the idea of using ultrasonic transducers for obtaining feedback signals proportional to the travelling time for the ultrasonic signals between the probe to a bone or bone part inside the human body, i.e. the cervical spine or a part of a cervical vertebra, having a substantially fixed position relative the tumor being treated. Motions of the bone or bone part, e.g. the spine or cervical vertebra, can be detected as changes in the received reflected signal. Hence, the present invention measures changes in the time intervals between the bone or part of bone and the transducers. Thereby, the measurements are performed directly on the bone or part of bone allowing a high degree of accuracy. Another advantage of the present invention is that the method and system for motion detection easily can be integrated or used together with radiation therapy systems, such as the Perfexion® system. The manufacturing cost for the motion monitoring system according to the present invention is low and it is easy and intuitive to use for the medical personnel. Since the motion monitoring method and system according to the present invention is non-invasive, i.e. measures motions of the patients without requiring any penetration of the skin of the patient, the system and method are comfortable for the patient and do not entail any risk for e.g. infections, which always is a risk when using invasive methods and systems.

According to embodiment of the present invention, distances to the bone or part of bone from the at least one ultrasonic transducer are calculated based on said determined time intervals and changes of the distances are detected, The motions of said bone or part of bone can be monitored using the detected changes of the distance, wherein said motions indicate that the patient or a part of the patient has moved. According to this embodiment of the present invention, feedback signals proportional to travelling time between the probe to the bone or bone part inside the human body, i.e. the cervical spine or a part of a cervical vertebra, are obtained and used to calculate a distance between the probe and the bone or part of bone. Motions of the bone or bone part, e.g. the spine or cervical vertebra, can be detected as changes in the distance.

According to embodiments of the present invention, an interrupting signal is provided instructing the radiation therapy system to interrupt the treatment if a motion change exceeding a predetermined limit and/or lasting at least a predetermined period of time is detected. Thereby, the treatment can be immediately interrupted if the patient has moved such that the therapy volume, e.g. a cancer tumor, has been moved from the initial treatment position, and hence potential damage to surrounding tissue can be avoided.

According to embodiments of the present invention, a tangible alert signal is provided if a motion change exceeding a predetermined limit and/or lasting at least a predetermined period of time is detected. Thereby, the medical personnel handling the radiation therapy system can be informed or notified that the patient has moved such that the therapy volume, e.g. a cancer tumor, has been moved from the initial treatment position, which hence may cause damage to surrounding tissue. The alert signal thus notifies the medical personnel that the therapy may have to be interrupted and the patient re-positioned before that therapy session is resumed. In embodiments of the present invention, the alert signal may be an audible signal or message and/or a visible signal or message.

According to embodiments of the present invention, a distance or time interval change is determined to be a detected change if the change exceeds a predetermined limit and/or lasts at least a predetermined time interval. Thereby, small motions associated, for example, with respiration that do not influence the therapy or cause damage to surrounding tissue can be filtered out and only motions that are large enough and/or are persistent are detected.

According to embodiments of the present invention, at least two ultrasonic transducer are positioned to generate ultrasound waves in a direction of a respective specific part of a bone being in a fixed relation to the cancer tumor such that the generated ultrasound waves are reflected by the respective specific part of bone. Further, time intervals are determined between generated ultrasound waves and corresponding sensed reflected ultrasound signals for each ultrasonic transducer using the reflected ultrasound waves at the respective ultrasonic transducer and a distance to the respective specific part of bone from each ultrasonic transducer is calculated based on the determined time intervals. Changes of the time intervals are detected, wherein a time interval change between an ultrasonic transducer and the respective specific part of bone represents a motion in one dimension, and motions in different dimensions of the bone or part of bone are monitored using the detected changes, wherein the motions indicate that the patient or a part of the patient has moved in one or more dimensions.

According to embodiments of the present invention, distances to the respective specific part of bone from each ultrasonic transducer are calculated based on the determined time intervals. Changes of each distance are detected, wherein a distance change between an ultrasonic transducer and the respective specific part of bone represents a motion in one dimension, and motions in different dimensions of the bone or part of bone are monitored using the detected changes of the distances, wherein the motions indicate that the patient or a part of the patient has moved in one or more dimensions.

According to embodiments of the present invention, the at least one ultrasonic transducer is configured to generate ultrasound waves having a frequency below about 4 MHz. Preferably, the at least one ultrasonic transducer is configured to generate ultrasound waves having a frequency within a frequency band of about 0.3-3.5 MHz, and more preferably within a frequency band of about 0.5-3 MHz.

According to embodiments of the present invention, the at least one ultrasonic transducer is integrated in a neck support structure for the patient and in embodiments the neck support structure is attached to the positioning system.

Further objects and advantages of the present invention will be discussed below by means of exemplifying embodiments.

These and other features, aspects and advantages of the invention will be more fully understood when considered with respect to the following detailed description, appended claims and accompanying drawings.

### BRIEF DESCRIPTION OF THE DRAWINGS

The drawings are not necessarily drawn to scale and illustrate generally, by way of example, but no way of limitation, various embodiments of the present invention. Thus, exemplifying embodiments of the invention are illustrated by way of example and not by way of limitation in the figures of the accompanying drawings in which like references indicate similar elements. It should be noted that references to “an” or “one” embodiment in this discussion are not necessarily to the same embodiment, and such references mean at least one.

Preferred embodiments of the invention will now be described in greater detail with reference to the accompanying drawings, in which

FIG. 1 schematically illustrates the general principle of a radiation therapy system in which the present invention is applicable;

FIG. 2 schematically illustrates an embodiment of a system according to the present invention;

FIG. 3a schematically illustrates a placement of an ultrasonic transducer according to the present invention;

FIG. 3b schematically illustrates an embodiment of a neck support structure with an ultrasonic transducer integrated used for achieving the placement shown in FIG. 3a;

FIG. 4a schematically illustrates another placement of an ultrasonic transducer according to the present invention;

FIG. 4b schematically illustrates an embodiment of a neck support structure in which two ultrasonic transducers are integrated used for achieving the placement shown in FIG. 4a;

FIG. 5 is a flow chart illustrating the general steps of a method according to the present invention;

FIG. 6 is a flow chart illustrating the steps of an embodiment of the method according to the present invention; and

FIG. 7 is a flow chart illustrating the steps of a further embodiment of the method according to the present invention.

### DESCRIPTION OF PREFERRED EMBODIMENTS

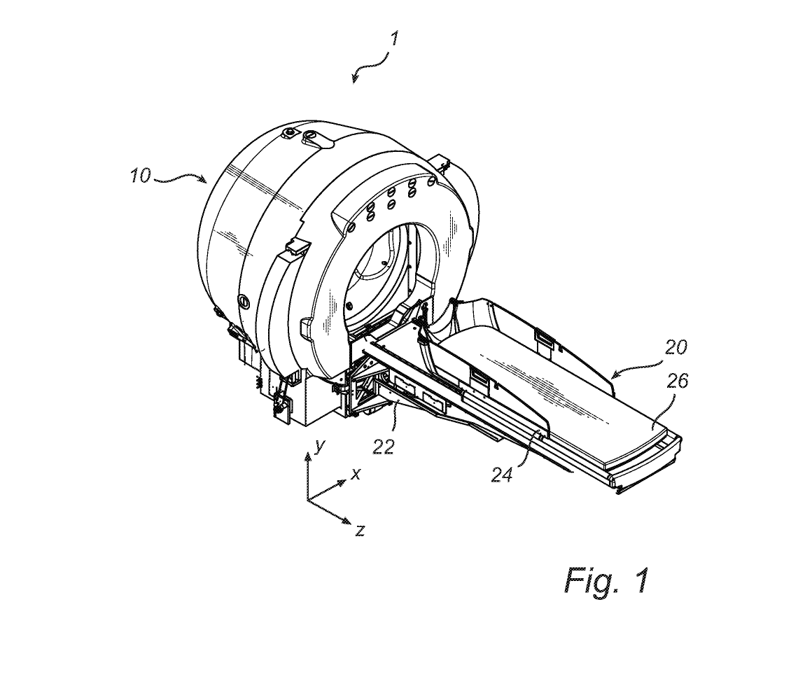

With reference to FIG. 1, a radiation therapy system 1 for which the present invention is applicable comprising a radiation unit 10 and a patient positioning unit 20 will be discussed. In the radiation unit 10, there are provided radioactive sources, radioactive source holders, a collimator body, and external shielding elements. The collimator body comprises a large number of collimator channels directed towards a common focus point, in a manner as is commonly known in the art.

The collimator body also acts as a radiation shield preventing radiation from reaching the patient other than through the collimator channels. Examples of collimator arrangements in radiation therapy systems applicable to the present invention can be found in WO 2004/06269 A1, which is hereby incorporated by reference. However, the present invention is also applicable to radiation therapy systems using other arrangements for collimating radiation into a fixed focus point, such as is disclosed in U.S. Pat. No. 4,780,898. The patient positioning unit 20 comprises a rigid framework 22, a slidable or movable carriage 24, and motors (not shown) for moving the carriage 24 in relation to the framework 22. The carriage 24 is further provided with a patient bed (not shown) for carrying and moving the entire patient. At one end of the carriage 24, there is provided a fixation arrangement (not shown) for receiving and fixing a stereotactic fixation unit (not show), either directly or via an adapter unit (not shown), and thereby preventing the stereotactic fixation unit from translational or rotational movement in relation to the movable carriage 24. The patient can be translated using the patient positioning unit 20 in the coordinate system of the radiation therapy system 1 or the patient positioning unit 20, e.g. along the three orthogonal axes x, y, and z shown in FIG. 1 or along axes in a polar coordinate system.

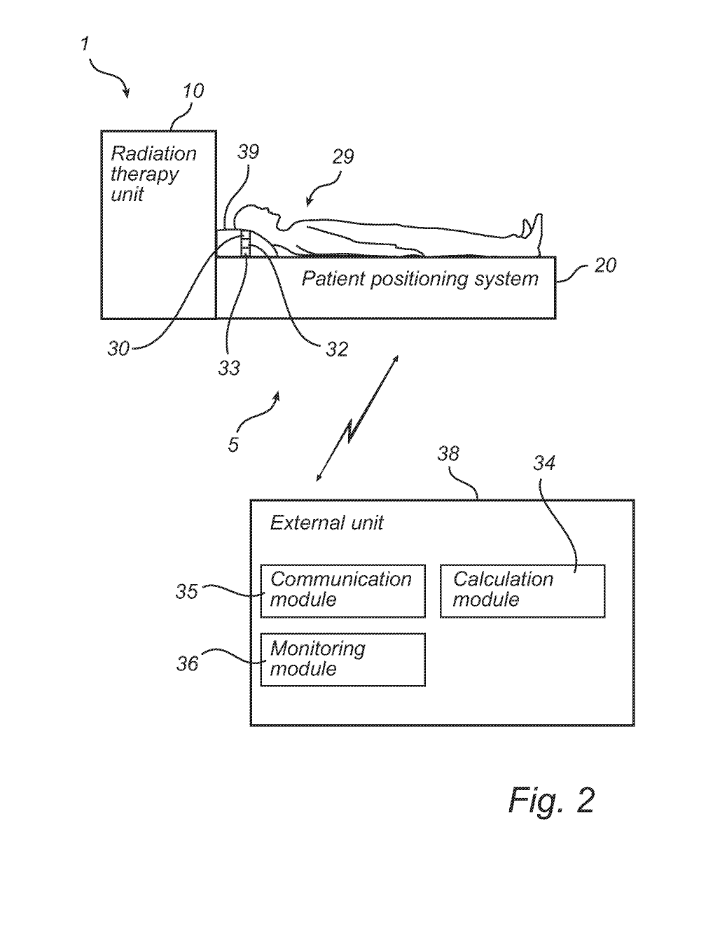

Turning now to FIG. 2, an embodiment of a system for intra-fraction motion detection and monitoring according to the present invention will be discussed. The intra-fraction motion detection system 5 comprises at least one ultrasonic transducer 30 positioned relative to the positioning system 20, preferably in contact with the patient 29 when placed in the positioning system 20, to generate ultrasound waves in a direction of a bone or a part of a bone being in a substantially fixed relation to the cancer tumor such that the generated ultrasound waves are reflected by the bone or part of bone. At least one ultrasonic transducer 30 is configured to sense the reflected ultrasound waves.

It is however also possible to generate the ultrasonic waves using a first ultrasonic transducer and to sense the reflected waves using a second ultrasonic transducer, or, several ultrasonic transducers.

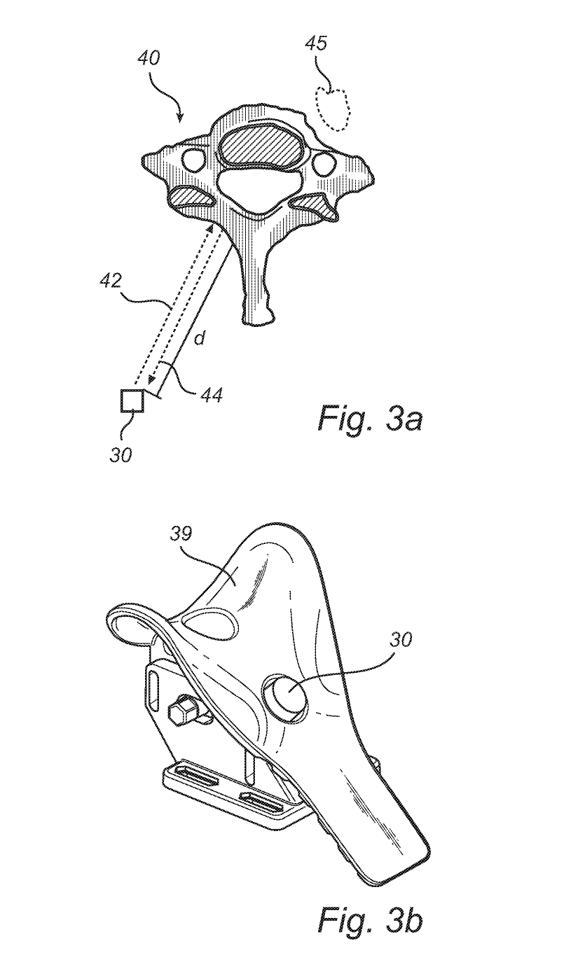

In FIG. 3a, a principle view of a placement of the ultrasonic transducer 30 close to cervical vertebra 40 of a patient having a tumor 45 in the cervical region is illustrated. The ultrasonic transducer 30 is integrated into a neck support structure 39 (see FIGS. 2 and 3b) arranged to be positioned in the patient position system 20 so as to support the head of the patient 29 during treatment. The ultrasonic transducer 30 generates ultrasound waves 42 that thereafter are reflected 44 by the cervical vertebra 40. Based on the time intervals between the generated signals 42 and the corresponding reflected signals 44, it is possible to detect the motions of the patient. In embodiments of the present invention, distances d between the ultrasonic transducer 30 and the cervical vertebra 40 are calculated using the time intervals and by observing and monitoring changes in the distance d, motions of the cervical vertebra 40 can be detected and, in turn, motions of the patient can be detected.

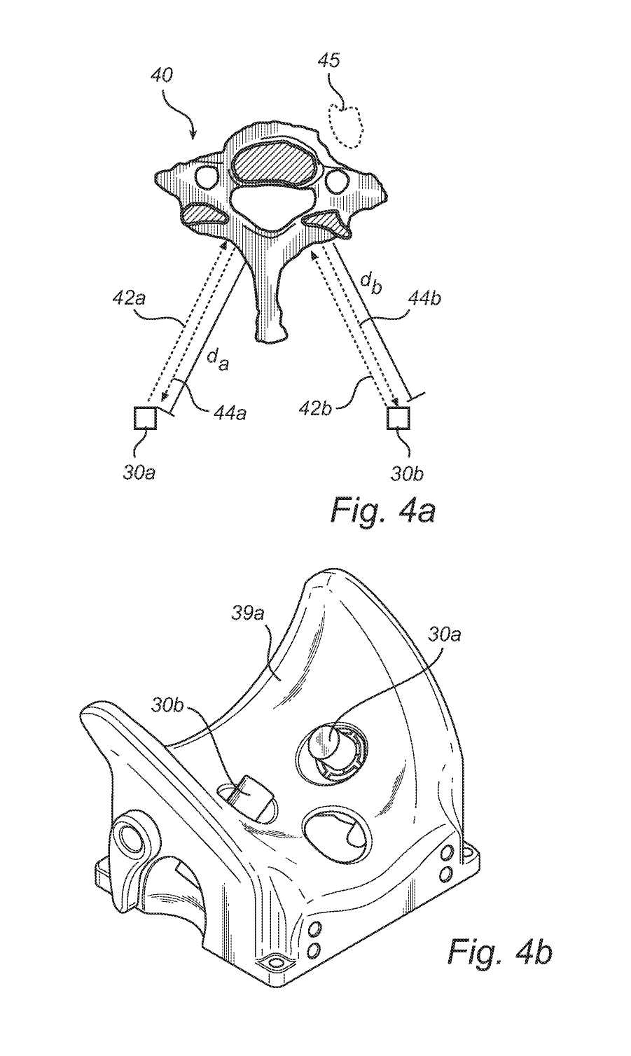

In FIG. 4a, another principle view of an arrangement of ultrasonic transducers 30a and 30b close to cervical vertebra 40 of a patient having a tumor 45 in the cervical region is illustrated. The ultrasonic transducers 30a and 30b are integrated into a neck support structure 39a (see FIG. 4b) arranged to be positioned in the patient position system 20 so as to support the head of the patient 29 during treatment. The ultrasonic transducers 30a and 30b generate ultrasound waves 42a and 42b, respectively, that thereafter are reflected 44a and 44b, respectively, by the cervical vertebra 40. Based on the respective time intervals between the generated signals 42a and 42b, respectively, and the corresponding reflected signals 44a and 44b, respectively, motions of the cervical vertebra 40 can be detected and, in turn, motions of the patient can be detected in two dimensions. In embodiments of the present invention, distances dₐ and d_{b} between the ultrasonic transducers 30a and 30b, respectively, and the cervical vertebra 40 are be calculated. By observing and monitoring changes in the distances dₐ and d_{b}, motions of the cervical vertebra 40 can be detected and, in turn, motions of the patient can be detected in two dimensions.

It is also conceivable to use for example three or more ultrasonic transducers. In FIG. 4a, the transducers 30a and 30b are placed and arranged such that the generated ultrasound waves impinge on different part of the cervical vertebra 40. However, it is also conceivable to arrange the transducers such that the generated ultrasound waves impinge on the same part of a bone (e.g. cervical vertebra). In such a solution, the transducers can be configured to generate the ultrasound waves in an alternating manner.

The intra-fraction motion detection system 5 further comprises a time interval determining module 32 configured to determine the time interval between generated ultrasound waves (e.g. 42 or 42a and 42b) and corresponding sensed reflected ultrasound signals (e.g. 44 or 44a and 44b).

In embodiments of the present invention, the time interval determining module 32 is integrated in the ultrasonic transducer 30.

A calculation module 34 is configured to calculate a distance to the bone or part of bone from the at least one ultrasonic transducer 30 based on the determined time interval.

A monitoring module 36 is configured to detect changes in the time intervals and/or distances and to monitor motions of the bone or part of bone using the detected changes of the time intervals and/or distances, wherein a motion indicates that the patient or a part of the patient has moved.

In embodiments of the present invention, the calculation module 34 and the monitoring module 36 are arranged in an external unit 38, for example, as software modules arranged to be executed on a computer unit.

Time interval data and/or data regarding generated and reflected ultrasound waves can be transferred to a communication module 35 of the external unit 38 from a communication module 33 of the ultrasonic transducer 30 wirelessly, e.g. using Bluetooth, or via cable.

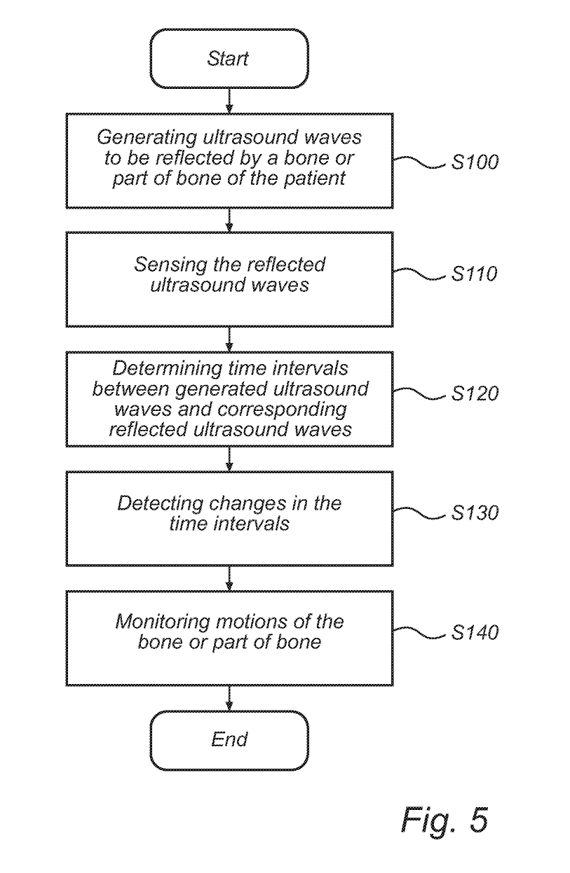

With reference now to FIG. 5, the general principles of a method according to the present invention will be discussed. The method can be used for intra-fraction motion detection and monitoring, for example, in therapy sessions during fractionated radiation therapy in connection with treatment of cancer such as treatment of cervical tumors. When the method has been initiated, the method is preferably continued during the whole treatment session so as to monitor patient movements throughout the session.

First, in step S100, after the patient has been positioned such that a treatment volume, e.g. the cancer tumor, is positioned in a treatment position in relation to the fixed focus point in the radiation therapy unit 10 and the patient has been placed on the patient positioning system 20 such that the ultrasonic transducer 30, 30a, and 30b is placed in correct position for delivering distance data, ultrasound waves 42, 42a and 42b are generated in a direction of a bone or a part of a bone 40 of the patient 29 being in a fixed relation to the cancer tumor 45 such that the generated ultrasound waves are reflected 44, 44a and 44b by the bone or part of bone 40.

At step S110, the corresponding reflected ultrasound waves 44, 44a and 44b are sensed at the ultrasonic transducer 30, 30a and 30b.

At step S120, time intervals between the generated ultrasound waves and corresponding sensed reflected ultrasound signals are determined in the time interval determining module 32.

At step S130, changes in the time intervals are detected and, in step S140, motions of the bone or part of bone are monitored in the monitoring module 36 using the detected changes, wherein the motions indicate whether the patient or a part of the patient has moved to determine whether the motions are large enough to influence the efficiency of the therapy.

The above procedure is repeated, i.e. steps S100-S140, until the treatment session is finished, terminated or interrupted.

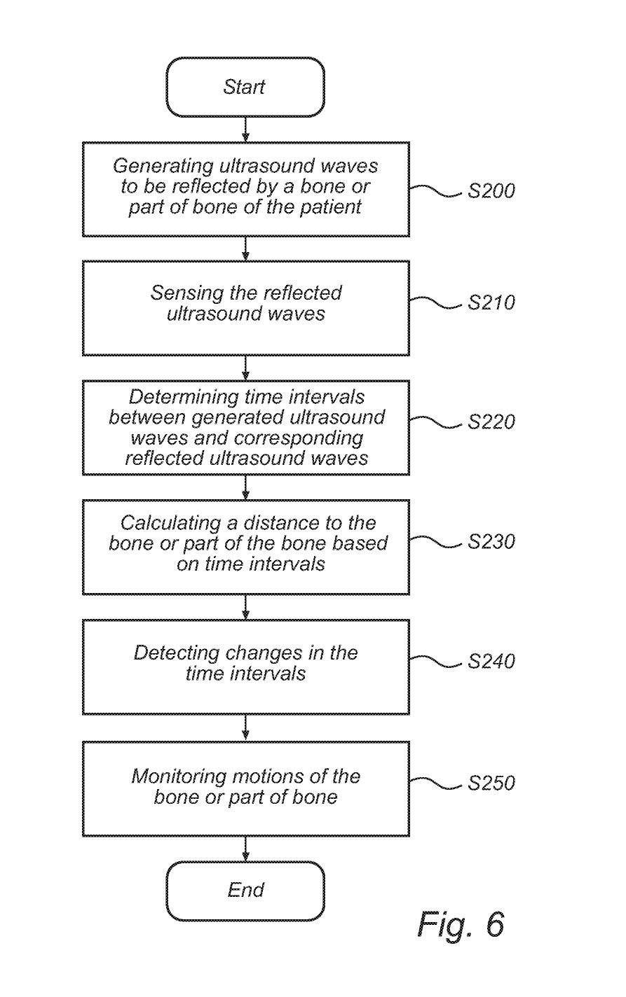

In FIG. 6, steps of an embodiment of the method according to the present invention are shown. First, in step S200, after the patient has been positioned such that a treatment volume, e.g. the cancer tumor, is positioned in a treatment position in relation to the fixed focus point in the radiation therapy unit 10 and the patient has been placed on the patient positioning system 20 such that the ultrasonic transducer 30, 30a, and 30b is placed in correct position for delivering distance data, ultrasound waves 42, 42a and 42b are generated in a direction of a bone or a part of a bone 40 of the patient 29 being in a fixed relation to the cancer tumor 45 such that the generated ultrasound waves are reflected 44, 44a and 44b by the bone or part of bone 40.

At step S210, the corresponding reflected ultrasound waves 44, 44a and 44b are sensed at the ultrasonic transducer 30, 30a and 30b.

At step S220, a time interval between the generated ultrasound wave and a corresponding sensed reflected ultrasound signal is determined in the time interval determining module 32.

Thereafter, at step S230, a distance d, dₐ and d_{b} to the bone or part of bone 40 from the ultrasonic transducer 30, 30a and 30b is calculated by the calculation module 34 based on the determined time interval.

Subsequently, at step S240, changes of the distance d, dₐ and d_{b} are detected in the monitoring module 36. Preferably, a distance change is determined to be a detected distance change if the change exceeds a predetermined limit and/or lasts at least a predetermined time interval. Thereby, very small and/or temporary motions that have no or very little impact on the treatment can be filtered out.

At step S250, motions of the bone or part of bone can be monitored in the monitoring module 36 using the detected changes of the distance d, dₐ and d_{b}, wherein the motions indicate whether the patient or a part of the patient has moved in an extent that the efficiency of the therapy may be impaired.

This procedure is repeated, i.e. steps S200-S250, until the treatment session is finished, terminated or interrupted.

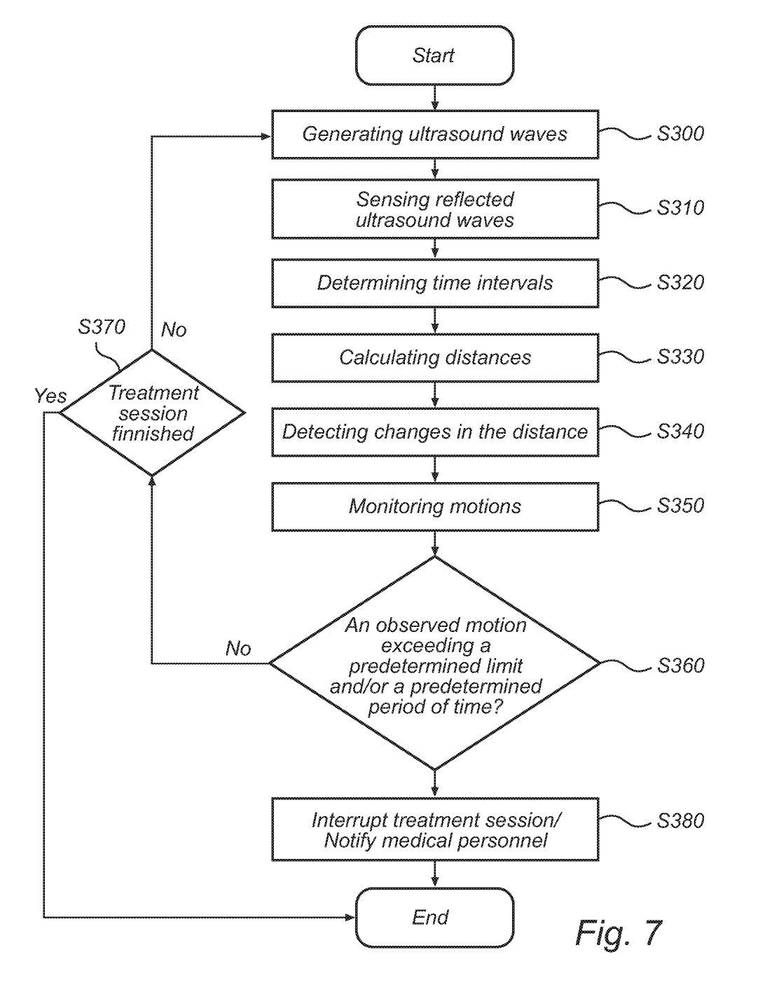

In FIG. 7, steps of a further embodiment of the method according present invention are shown.

In step S300, after the patient has been positioned such that a treatment volume, e.g. the cancer tumor, is positioned in a treatment position in relation to the fixed focus point in the radiation therapy unit 10 and the patient has been placed on the patient positioning system 20 such that the ultrasonic transducer 30, 30a, and 30b is placed in correct position for delivering distance data, ultrasound waves 42, 42a and 42b are generated in a direction of a bone or a part of a bone 40 of the patient 29 being in a fixed relation to the cancer tumor 45 such that the generated ultrasound waves are reflected 44, 44a and 44b by the bone or part of bone 40.

At step S310, the corresponding reflected ultrasound waves 44, 44a and 44b are sensed at the ultrasonic transducer 30, 30a and 30b.

At step S320, a time interval between the generated ultrasound wave and a corresponding sensed reflected ultrasound signal is determined in the time interval determining module 32.

Thereafter, at step 330, a distance d, dₐ and d_{b} to the bone or part of bone 40 from the ultrasonic transducer 30, 30a and 30b is calculated by the calculation module 34 based on the determined time interval.

Subsequently, at step S340, changes of the distance d, dₐ and d_{b} are detected in the monitoring module 36. Preferably, a distance change is determined to be a detected distance change if the change exceeds a predetermined limit and/or lasts at least a predetermined time interval. Thereby, very small and/or temporary motions that have no or very little impact on the treatment can be filtered out.

At step S350, motions of the bone or part of bone can be monitored in the monitoring module 36 using the detected changes of the distance d, dₐ and d_{b}, wherein the motions indicate whether the patient or a part of the patient has moved in an extent that the efficiency of the therapy may be impaired.

In step S360, it is checked whether an observed motion exceeds a predetermined limit and/or lasts at least a predetermined period of time. If no, and if the treatment session is still in process (step S370), new ultrasound pulses are generated in step S300. On the other hand, if a motion is observed that exceeds the predetermined limit and/or lasts the predetermined period of time, the treatment session is interrupted and/or an alert signal is issued in step S380.

The monitoring module 36 may send an interruption signal to the radiation therapy system 1 instructing it to immediately interrupt the treatment. Thereby, it is secured that potential damage to surrounding tissue is minimized.

If an alert signal is issued, the alert signal may be an audible and/or visible signal. Thereby, the medical personnel performing the therapy is informed and alerted of the fact that the patient has moved from its initial therapy position, which may lead to impaired therapy, and may take proper actions.

Even though the present invention has been described above using exemplifying embodiments thereof, alterations, modifications, and combinations thereof, as understood by those skilled in the art, may be made without departing from the scope of the invention as defined in the accompanying claims.

1. A method for monitoring intra-fraction motions of a patient in connection with treatment of treatment volumes such as cancer tumors of said patient in a radiation therapy system, which radiation therapy system comprises a radiation therapy unit having a fixed radiation focus point, and a patient positioning system for positioning a treatment volume in a patient in relation to said fixed focus point in the radiation therapy unit, said method comprising:
generating ultrasound waves in a direction of a bone or a part of a bone of said patient being in a fixed relation to said treatment volume such that said generated ultrasound waves are reflected by said bone or part of bone using at least one ultrasonic transducer;
sensing the reflected ultrasound waves using at least one ultrasonic transducer;
determining time intervals between generated ultrasound waves and corresponding sensed reflected ultrasound signals for said at least one ultrasonic transducer; and
monitoring motions of said bone or part of bone using said time intervals, wherein said motions indicate that the patient or a part of said patient has moved.

2. The method according to claim 1, further comprising the steps of:
calculating a distance to said bone or part of bone from said at least one ultrasonic transducer based on said determined time intervals;
detecting changes of said distance; and
monitoring motions of said bone or part of bone using said detected changes of said distance, wherein said motions indicate that the patient or a part of said patient has moved.

3. The method according to claim 1, wherein said step of detecting comprises determining a time interval change or distance to be a detected change if the change exceeds a predetermined limit and/or lasts at least a predetermined period of time.

4. The method according to claim 1, further comprising providing an interrupting signal instructing the radiation therapy system to interrupt the treatment if a motion change exceeding a predetermined limit and/or lasting at least a predetermined period of time is detected

5. The method according to claim 1, further comprising providing a tangible alert signal if a motion change exceeding a predetermined limit and/or lasting at least a predetermined period of time is detected.

6. The method according to claim 1, wherein the step of positioning at least one ultrasonic transducer comprises
positioning at least two ultrasonic transducer to generate ultrasound waves in a direction of a respective specific part of a bone being in a fixed relation to said cancer tumor such that said generated ultrasound waves are reflected by said respective specific part of bone.

7. The method according to claim 6, further comprising:
sensing the reflected ultrasound waves at the respective ultrasonic transducer;
determining time intervals between generated ultrasound waves and corresponding sensed reflected ultrasound signals for each ultrasonic transducer; and
monitoring motions in different dimensions of said bone or part of bone using said time intervals, wherein said motions indicate that the patient or a part of said patient has moved in one or more dimensions.

8. The method according to claim 7, further comprising:
calculating a distance to said respective specific part of bone from each ultrasonic transducer based on said determined time intervals;
detecting changes of each distance, wherein a distance change between an ultrasonic transducer and the respective specific part of bone represents a motion in one dimension; and
monitoring motions in different dimensions of said bone or part of bone using said detected distances, wherein said motions indicate that the patient or a part of said patient has moved in one or more dimensions.

9. A system for monitoring intra-fraction motions of a patient in connection with treatment of treatment volumes such as cancer tumors of said patient in a radiation therapy system, which radiation therapy system comprises a radiation therapy unit having a fixed radiation focus point, a patient positioning system for positioning a treatment volume in a patient in relation to said fixed focus point in the radiation therapy unit, said system comprising:
at least one ultrasonic transducer positioned relative to said positioning system to generate ultrasound waves in a direction of a bone or a part of a bone being in a fixed relation to said treatment volume such that said generated ultrasound waves are reflected by said bone or part of bone, wherein a sensor of at least one ultrasonic transducer is configured to sense the reflected ultrasound waves;
a time interval determining module configured to determine the time intervals between generated ultrasound waves and corresponding sensed reflected ultrasound signals for said at least one ultrasonic transducer; and
a monitoring module configured to detect changes in said time intervals and to monitor motions of said bone or part of bone using said detected changes of the time intervals, wherein a motion indicates that the patient or a part of said patient has moved.

10. The system according to claim 9, further comprising:
a calculation module configured to calculate a distance to said bone or part of bone from at least one ultrasonic transducer based on said determined time interval; and wherein
said monitoring module is configured to detect changes in said distance and to monitor motions of said bone or part of bone using said detected changes of said distance, wherein a motion indicates that the patient or a part of said patient has moved.

11. The system according to claim 9, wherein said monitoring module is configured to determine a time interval change or a distance change to be a detected change if the change exceeds a predetermined limit and/or lasts at least a predetermined period of time.

12. The system according to claim 9, wherein said monitoring module is configured to provide an interruption signal instructing said radiation therapy system to interrupt said treatment if a motion change exceeding a predetermined limit and/or lasting at least predetermined period of time is detected.

13. The system according to claim 9, wherein said monitoring module is configured to provide an alert signal if a motion change exceeding a predetermined limit and/or lasting at least predetermined period of time is detected.

14. The system according to claim 9, further comprises:
at least two ultrasonic transducer positioned to generate ultrasound waves in a direction of a respective specific part of a bone being in a fixed relation to said cancer tumor such that said generated ultrasound waves are reflected by said respective specific part of bone.

15. The system according to claim 14, wherein:
said time interval determining module is configured to determine time intervals between generated ultrasound waves and corresponding sensed reflected ultrasound signals for each ultrasonic transducer; and
said monitoring module is configured to detect changes of each time interval, wherein a time interval change between an ultrasonic transducer and the respective specific part of bone represents a motion in one dimension, and to monitor motions in different dimensions of said bone or part of bone using said detected changes of said time intervals, wherein said motions indicate that the patient or a part of said patient has moved in one or more dimensions.

16. The system according to claim 15, wherein:
said calculation module is configured to calculate a distance to said respective specific part of bone from each ultrasonic transducer based on said determined time intervals; and wherein
said monitoring module is configured to detect changes of each distance, wherein a distance change between an ultrasonic transducer and the respective specific part of bone represents a motion in one dimension, and to monitor motions in different dimensions of said bone or part of bone using said detected changes of said distances, wherein said motions indicate that the patient or a part of said patient has moved in one or more dimensions.

17. The system according to claim 9, wherein said at least one ultrasonic transducer is integrated in a neck support structure for said patient.

18. The system according to claim 17, wherein said neck support structure is arranged to be attached to said positioning system.

19. The method according to claim 2, wherein said step of detecting comprises determining a time interval change or distance to be a detected change if the change exceeds a predetermined limit and/or lasts at least a predetermined period of time.

20. The method according to claim 2, further comprising providing an interrupting signal instructing the radiation therapy system to interrupt the treatment if a motion change exceeding a predetermined limit and/or lasting at least a predetermined period of time is detected
